(19) United States
(12) Patent Application Publication
(10) Pub. No.: US 2014/0227558 A1
(43) Pub. Date: Aug. 14, 2014

(54) CATHETER WIRE AND METHOD OF MANUFACTURING THE SAME

(71) Applicant: HITACHI CABLE, LTD., Tokyo (JP)

(72) Inventors: Takanobu WATANABE, Hitachi (JP); Detian HUANG, Hitachi (JP)

(73) Assignee: HITACHI CABLE, LTD., Tokyo (JP)

(21) Appl. No.: 13/868,358

(22) Filed: Apr. 23, 2013

(30) Foreign Application Priority Data
Feb. 14, 2013 (JP) 2013-026686

(51) Int. Cl. A61L 29/08; A61L 29/02

(52) U.S. Cl. CPC A61L 29/085; A61L 29/02
USPC 428/685; 428/457; 29/527.4; 205/151

(57) ABSTRACT

A catheter wire includes a wire core including a semi-rigid stainless steel, and a conductor layer covering an outer periphery of the wire core. A method of manufacturing a catheter wire includes drawing a stainless steel in an axial direction so as to form a wire core with a predetermined diameter, annealing the drawn stainless steel so as to change the stainless steel into a semi-rigid stainless steel, and forming a conductor layer on an outer periphery of the semi-rigid stainless steel.

The present application is based on Japanese patent application No. 2013-026686 filed on Feb. 14, 2013, the entire contents of which are incorporated herein by reference.

### BACKGROUND OF THE INVENTION

1. Field of the Invention

The invention relates to a catheter wire and a method of manufacturing the catheter wire and, in particular, to a catheter wire that is good in tensile strength and electrical characteristics in a longitudinal direction and is less likely to be broken during welding to connect an electrode thereto, and a method of manufacturing the catheter wire.

2. Description of the Related Art

A conventional catheter wire is formed of a cladding material provided with a wire core 100 formed of hard stainless steel (hereinafter, referred to as “SUS”) or iron (hereinafter, referred to as “Fe”) and a conductor layer 110 formed of copper covering an outer periphery of the wire core 100, as shown in FIG. 3A. Meanwhile, a catheter wire using a core formed of a copper alloy 120 as shown in FIG. 3B is also known.

For example, JP-A-2004-194768 discloses a straight wire formed of a high-strength stainless steel as a guide wire core of medical catheter.

### SUMMARY OF THE INVENTION

The cladding material shown in FIG. 3A is drawn and is thinned so as to have a predetermined outer diameter (e.g., 0.3 mm) which is desirable as a catheter wire. Since elongation of hard SUS or Fe forming the wire core 100 is different from that of copper forming the conductor layer 110, a cross-sectional area ratio of the wire core 100 to the conductor layer 110 varies in a longitudinal direction when the wire is drawn.

As a result, a problem arises in that the cross-sectional area of the wire core 100 formed of hard SUS or Fe is non-uniform, resulting in unstable tensile strength in a longitudinal direction. In addition, there is also a problem that the cross-sectional area of the copper forming the conductor layer 110 is also non-uniform, resulting in unstable electrical characteristics in a longitudinal direction.

In addition, a conventional cladding material has an enamel layer (not shown) which covers an outer periphery of the conductor layer 110. Therefore, dangerous work using hot sodium hydroxide to dissolve and remove the enamel layer is required at the time of terminal processing to connect an electrode and this causes significant deterioration in workability of connecting the electrode.

In addition, when heat of welding is applied to the wire disclosed in JP-A-2004-194768 at the time of terminal processing to connect the electrode, a heated portion (hereinafter, referred to as a “welded portion”) becomes annealed. Accordingly, the welded portion becomes much softer than a non-welded portion which is not heated, and this causes a problem that the wire is likely to be broken at an interface between the non-welded portion and the welded portion.

Furthermore, strength may not be sufficient in the structure shown in FIG. 3B in which only a core formed of copper alloy is used.

It is an object of the invention to provide a catheter wire that is good in tensile strength and electrical characteristics in a longitudinal direction and is less likely to be broken during welding to connect an electrode thereto, as well as a method of manufacturing the catheter wire.
(1) According to one embodiment of the invention, a catheter wire comprises:
   a wire core comprising a semi-rigid stainless steel; and
   a conductor layer covering an outer periphery of the wire core.

In the above embodiment (1) of the invention, the following modifications and changes can be made.
(i) The conductor layer comprises a plated layer with a conductive metal plated.
(ii) The semi-rigid stainless steel has a tensile strength at break of not less than 1500 MPa and not more than 2000 MPa.
(iii) The catheter wire further comprises a resin layer covering an outer periphery of the conductor layer.
(iv) The catheter wire has a tensile breaking load of not less than 3N and not more than 7N, and a DC resistance of not more than 15 Ω/m.
(2) According to another embodiment of the invention, a method of manufacturing a catheter wire comprises:
   drawing a stainless steel in an axial direction so as to form a wire core with a predetermined diameter;
   annealing the drawn stainless steel so as to change the stainless steel into a semi-rigid stainless steel; and
   forming a conductor layer on an outer periphery of the semi-rigid stainless steel.

In the above embodiment (2) of the invention, the following modifications and changes can be made.
(v) The forming of the conductor layer comprises plating a conductive metal on the semi-rigid stainless steel.
(vi) The method further comprises forming a resin layer on an outer periphery of the conductor layer.
(vii) The annealing comprises annealing the stainless steel into the semi-rigid stainless steel having a tensile strength at break of not less than 1500 MPa and not more than 2000 MPa.

### EFFECTS OF THE INVENTION

According to one embodiment of the invention, a catheter wire can be provided that is good in tensile strength and electrical characteristics in a longitudinal direction and is less likely to be broken during welding to connect an electrode thereto, as well as a method of manufacturing the catheter wire.

### BRIEF DESCRIPTION OF THE DRAWINGS

Next, the present invention will be explained in more detail in conjunction with appended drawings, wherein:

FIG. 1 is a schematic cross sectional view showing a catheter wire in an embodiment of the present invention;

FIG. 2 is a flow chart showing a method of manufacturing the catheter wire in the embodiment of the invention; and

FIGS. 3A and 3B are schematic cross sectional views showing a conventional catheter wire.

### DETAILED DESCRIPTION OF THE PREFERRED EMBODIMENTS

A catheter wire and a method of manufacturing the same in the embodiment of the invention will be described below in conjunction with the drawings.

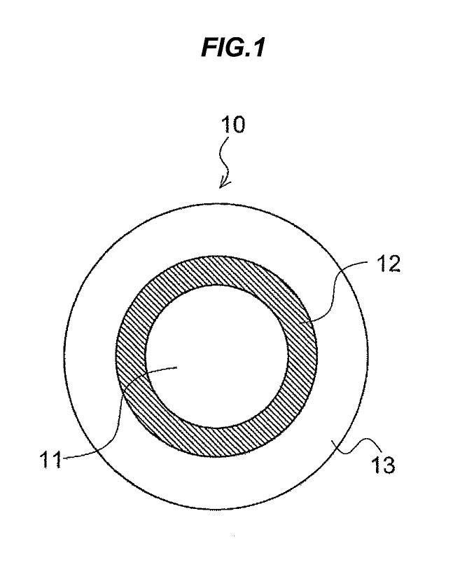

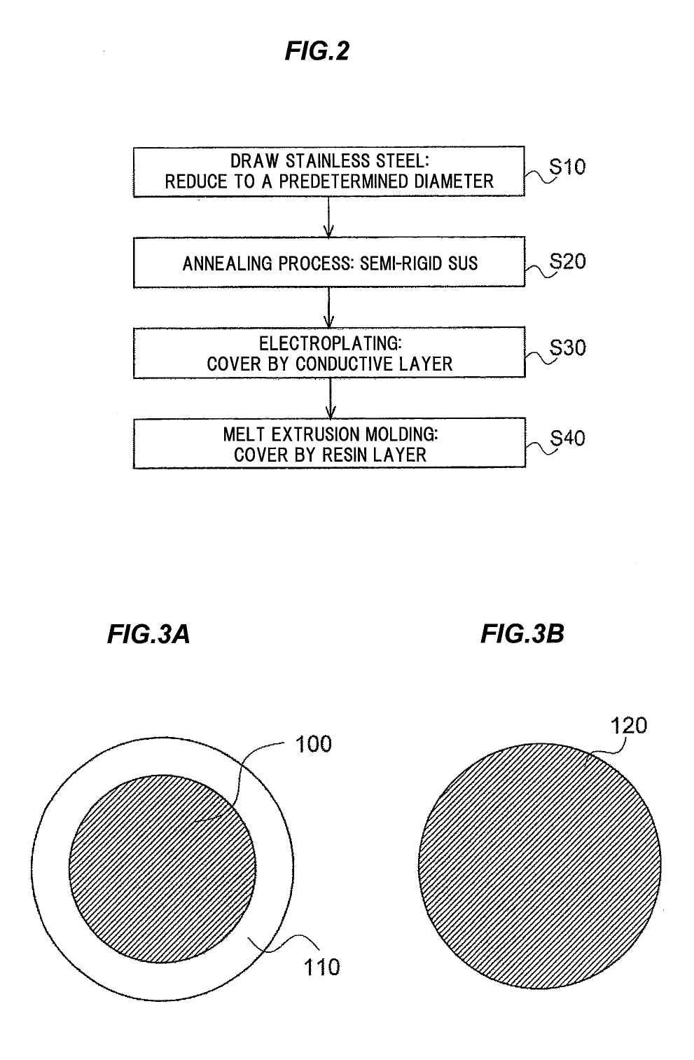

As shown in FIG. 1, a catheter wire 10 has a wire core 11 formed of a semi-rigid stainless steel (hereinafter, referred to as “semi-rigid SUS”), a conductor layer 12 covering an outer periphery of the wire core 11 and a resin layer 13 covering an outer periphery of the conductor layer 12.

The semi-rigid SUS forming the wire core 11 is a stainless steel which is drawn in a longitudinal direction (axial direction) so as to have a predetermined outer diameter and is then annealed. Use of the semi-rigid SUS for the wire core 11 as described above suppresses composition change in a welded portion even when heat of electric welding is applied to the catheter wire 10 at the time of terminal processing to connect an electrode (not shown). As a result, the wire core 11 has a small difference in hardness between the welded portion and a non-welded portion and it is thus possible to efficiently prevent breakage from occurring at an interface between the welded portion and the non-welded portion.

In the present embodiment, a diameter of the wire core 11 is about 0.06 mm from the viewpoint of reduction in diameter. In addition, a tensile strength at break of the semi-rigid SUS is not less than 1500 MPa and not more than 2000 MPa from the viewpoint of maintaining strength.

The conductor layer 12 is obtained by plating a metal excellent in conductivity, e.g., copper or silver, so as to cover the outer periphery of the wire core 11. Covering with the conductive metal by plating as described above eliminates the need of wire drawing performed on a conventional cladding material and allows the conductor layer 12 with a uniform thickness to be formed. As a result, a cross-sectional area ratio of the wire core 11 to the conductor layer 12 is uniform throughout and this allows tensile strength and electrical characteristics in a longitudinal direction to be effectively stabilized.

In the present embodiment, the thickness of the conductor layer 12 is about 6 μm from the viewpoint of reduction in diameter and electrical characteristics.

The resin layer 13 is formed of, e.g., a fluorine resin such as tetrafluoroethylene perfluoroalkyl vinyl ether copolymer (PFA) which is melt-extrusion molded so as to cover an outer periphery of the conductor layer 12. Use of the fluorine resin for the resin layer 13 as described above allows the resin layer 13 to be easily removed by a tool such as wire stripper at the time of terminal processing to connect an electrode (not shown) to the catheter wire 10. As a result, dangerous work using a chemical to remove an enamel layer, which is the work performed on the conventional cladding material, is no longer necessary and it is thus possible to effectively improve workability of connecting the electrode and safety.

In the present embodiment, the thickness of the resin layer 13 (the fluorine resin) is about 15 μm from the viewpoint of reduction in diameter. In addition, a melt flow rate (MFR) of the fluorine resin is not less than 30 so that good fluidity is provided at the time of extrusion molding.

In the catheter wire 10 of the present embodiment configured as described above, the diameter of the wire core 11 is about 0.06 mm, the thickness of the conductor layer 12 is 6 μm and the thickness of the resin layer 13 is 15 μm. Therefore, the catheter wire 10 has a reduced outer diameter of not more than 0.12 mm and a reduced DC resistance of not more than 15 Ω/m. In addition, since the tensile strength at break of the semi-rigid SUS is not less than 1500 MPa and not more than 2000 MPa, it is ensured that the total tensile breaking load of the wire core 11, the conductor layer 12 and the resin layer 13 is not less than 3N and not more than 7N.

Next, a method of manufacturing the catheter wire 10 in the present embodiment will be described in conjunction with FIG. 2.

In Step 10 (hereinafter, “step” is simply denoted by “S”), a stainless steel for forming the wire core 11 is drawn in a longitudinal direction to reduce a diameter thereof to a predetermined diameter (a diameter of about 0.06 mm in the present embodiment).

In S20, the drawn stainless steel is annealed so as to be transformed into a semi-rigid SUS having a tensile strength at break of not less than 1500 MPa and not more than 2000 MPa.

In S30, electroplating is performed so that a conductive metal (e.g., silver or copper) as the conductor layer 12 with a thickness of about 6 μm covers an outer periphery of the semi-rigid SUS.

In S40, melt extrusion molding is performed so that a resin layer (e.g., PFA) with a thickness of 15 μm covers on an outer periphery of the conductor layer 12.

In the catheter wire 10 obtained as described above, composition change due to welding heat during the terminal processing is reduced by transforming the wire core 11 into the semi-rigid SUS in the annealing process (S20) and it is thereby possible to prevent breakage from occurring at the interface between the welded portion and the non-welded portion. In addition, since the plating process is performed to cover the semi-rigid SUS with the conductor layer 12 (S30), a cross-sectional area ratio of the wire core 11 to the conductor layer 12 is uniform throughout, resulting in that tensile strength and electrical characteristics respectively in a longitudinal direction are good. Next, operations and effects of the catheter wire 10 in the present embodiment will be described.

In the conventional cladding material, hard SUS is used as a wire core. Therefore, a difference in hardness between the welded portion and the non-welded portion becomes large when heat of electric welding is applied at the time of terminal processing to connect an electrode and breakage is thus likely to occur at the interface between the welded portion and the non-welded portion.

On the other hand, in the catheter wire 10 of the present embodiment, the semi-rigid SUS formed by annealing stainless steel is used as the wire core 11. That is, composition change in the welded portion of the wire core 11 is suppressed even when heat of electric welding is applied at the time of terminal processing to connect an electrode.

Accordingly, in the catheter wire 10 of the present embodiment, the wire core 11 has a small difference in hardness between the welded portion and the non-welded portion and it is thus possible to efficiently prevent breakage from occurring at the interface between the welded portion and the non-welded portion.

Meanwhile, wire drawing is performed on the conventional cladding material. Therefore, there is a problem that a cross-sectional area ratio of the wire core to the conductor layer is non-uniform due to a difference in elongation between different metals (hard SUS and copper), resulting in that tensile strength and electrical characteristics in a longitudinal direction become unstable.

On the other hand, in the catheter wire 10 of the present embodiment, since the outer periphery of the wire core 11 formed of the semi-rigid SUS is covered with the conductive metal (silver or copper) by electroplating without performing the wire drawing, it is possible to form the conductor layer 12 with a uniform thickness.

Therefore, in the catheter wire 10 of the present embodiment, a cross-sectional area ratio of the wire core 11 to the conductor layer 12 is uniform throughout and it is thereby possible to effectively stabilize tensile strength and electrical characteristics in a longitudinal direction.

In the conventional cladding material, dangerous work using hot sodium hydroxide to dissolve and remove the enamel layer on the surface is required at the time of terminal processing to connect an electrode.

On the other hand, in the catheter wire 10 of the present embodiment, melt extrusion molding is performed to cover the outer periphery of the conductor layer 12 with the resin layer 13 formed of the fluorine resin. In other words, it is configured to allow the resin layer 13 to be easily removed by a tool such as wire stripper at the time of terminal processing to connect an electrode.

Therefore, in the catheter wire 10 of the present embodiment, dangerous work using a chemical to remove the enamel layer, which is the work performed on the conventional cladding material, is no longer necessary and it is thus possible to significantly improve workability of connecting the electrode and safety.

The present invention is not intended to be limited to the above-mentioned embodiment and can be appropriately modified and implemented without departing from the gist of the invention.

For example, tensile strength at break of the semi-rigid SUS, a diameter of the wire core 11 and thicknesses of the conductor layer 12 and the resin layer 13 are not limited to the above-mentioned numerical values and can be appropriately changed to optimal numerical values depending on the intended use or technical specification. In addition, the resin layer 13 is not limited to the fluorine resin and it is possible to use other resins.

What is claimed is:

1. A catheter wire, comprising:
a wire core comprising a semi-rigid stainless steel; and
a conductor layer covering an outer periphery of the wire core.

2. The catheter wire according to claim 1, wherein the conductor layer comprises a plated layer with a conductive metal plated.

3. The catheter wire according to claim 1, wherein the semi-rigid stainless steel has a tensile strength at break of not less than 1500 MPa and not more than 2000 MPa.

4. The catheter wire according to claim 1, further comprising a resin layer covering an outer periphery of the conductor layer.

5. The catheter wire according to claim 4, wherein the catheter wire has a tensile breaking load of not less than 3N and not more than 7N, and a DC resistance of not more than 15 Ω/m.

6. A method of manufacturing a catheter wire, comprising:
drawing a stainless steel in an axial direction so as to form a wire core with a predetermined diameter;
annealing the drawn stainless steel so as to change the stainless steel into a semi-rigid stainless steel; and
forming a conductor layer on an outer periphery of the semi-rigid stainless steel.

7. The method according to claim 6, wherein the forming of the conductor layer comprises plating a conductive metal on the semi-rigid stainless steel.

8. The method according to claim 6, further comprising forming a resin layer on an outer periphery of the conductor layer.

9. The method according to claim 6, wherein the annealing comprises annealing the stainless steel into the semi-rigid stainless steel having a tensile strength at break of not less than 1500 MPa and not more than 2000 MPa.
